(19) United States
(12) Patent Application Publication
(10) Pub. No.: US 2026/0221051 A1
(43) Pub. Date: Jul. 30, 2026

(54) METHOD OF INCREMENTAL TRAINING TO CREATE NEW PATTERNS OF PHYSIOLOGICAL CONTROL SIGNALS

(71) Applicants: CARNEGIE MELLON UNIVERSITY, Pittsburgh, PA (US); UNIVERSITY OF PITTSBURGH – OF THE COMMONWEALTH SYSTEM OF HIGHER EDUCATION, Pittsburgh, PA (US)

(72) Inventors: Emily R. Oby, Pittsburgh, PA (US); Aaron P. Batista, Pittsburgh, PA (US); Steven M. Chase, Pittsburgh, PA (US); Alan D. Degenhart, Pittsburgh, PA (US); Matthew D. Golub, Stanford, CA (US); Patrick T. Sadtler, Pittsburgh, PA (US); Byron M. Yu, Pittsburgh, PA (US)

(73) Assignees: CARNEGIE MELLON UNIVERSITY, Pittsburgh, PA (US); UNIVERSITY OF PITTSBURGH – OF THE COMMONWEALTH SYSTEM OF HIGHER EDUCATION, Pittsburgh, PA (US)

(21) Appl. No.: 19/574,953

(22) Filed: Mar. 23, 2026

Related U.S. Application Data
(63) Continuation of application No. 17/343,050, filed on Jun. 9, 2021, now abandoned.
(60) Provisional application No. 63/036,735, filed on Jun. 9, 2020.

(51) Int. Cl. G09B 19/00; A61B 5/372

(52) U.S. Cl. CPC G09B 19/00; A61B 5/372; A61B 2505/09

(57) ABSTRACT

Disclosed herein is a method for training a subject to produce a new neural activity pattern that results in a desired behavior. The method creates a brain-computer interface mapping between a neural activity pattern of a set of neural units and the desired behavior in an intrinsic manifold, without learning. An outside manifold perturbation of the mapping is then created, defining a new neural activity pattern lying outside of the intrinsic manifold that will produce the desired behavior. The new neural activity pattern is taught by incrementally perturbing the neural activity pattern that produces the desired behavior between the intrinsic manifold and the outside manifold perturbation and having the subject learn the desired behavior for each increment.

### RELATED APPLICATIONS

This application is a continuation of U.S. patent application Ser. No. 17/343,050, filed Jun. 9, 2021, which claims the benefit of U.S. Provisional Patent Application No. 63/036,735, filed Jun. 9, 2020, the contents of which are incorporated herein in their entirety.

### GOVERNMENT INTEREST

This invention was made with U.S. Government support under contract R01 HD071686 awarded by the National Institutes of Health and contract BCS1533672, awarded by the National Science Foundation. The U.S. Government has certain rights in this invention.

### BACKGROUND

A major, lucrative area for companies in the coming years is devices that interface with the central or peripheral nervous system. For example, companies are now developing devices to read the “thoughts” of a subject or to read the “movement intentions” of a subject, and then to enact those commands by moving the subject's own limb (via muscle stimulation), a robotic limb, a computer cursor, a computer game character, etc. These technologies can be used for rehabilitation (e.g., treatment for stroke), for mood regulation, or for enhancing a person's current capabilities.

In many, if not most of these applications, the subject's brain will need to be trained to interact with the device to appropriately control the effector. The engineers building user interfaces may wish to ascribe arbitrary functions of the device to new neural activity patterns, thereby expanding the subject's current capacities.

Learning has been associated with changes in the brain at every level of organization. However, it remains difficult to establish a causal link between specific changes in the brain and new behavioral abilities. The structure of neural population activity limits the learning that can occur within a single day. How neurons naturally covary can be characterized as the “intrinsic manifold”. Using a brain-computer interface (BCI) system, neural activity is mapped into the intrinsic manifold and then to a behavior. A BCI mapping that is consistent with the intrinsic manifold (i.e., a “within-manifold perturbation” (WMP)), can be learned quickly, usually within a single day. The neural strategy for learning WMPs does not involve the formation of new neural activity patterns. Instead, learning occurs by reassociating preexisting patterns of neural activity with different intended behaviors.

However, it would be desirable to be able to create new neural activity patterns consistent with BCI mappings outside of the intrinsic manifold (i.e., “outside manifold perturbations” (OMPs)), which would encourage the formation of new neural activity patterns which may be associated with new intended behaviors.

### BRIEF DESCRIPTION OF THE DRAWINGS

FIG. 1 is a graphical depiction of the incremental training approach of the present invention.

FIG. 2 summarizes the training scheme used for single- and multi-day experiments with and without incremental training, respectively, wherein each block indicates a day.

FIGS. 3A-3B are graphs showing the learning curves for multi-day OMP incremental training for two subject monkeys.

FIGS. 4A-4B are graphs showing the learning curves for multi-day OMP training, with no incremental training for two subject monkeys.

FIGS. 5A-5C are graphs of histograms quantifying the amount of learning for single-day OMP training, multiday OMP (with incremental) training and single-day WMP training, respectively.

FIGS. 6A-6C are schematic depictions of the technique used to identify outside-repertoire neural activity patterns.

FIGS. 7A-7D are graphs showing that new neural activity patterns move progressively further away from the speed limit with incremental learning. FIG. 7E is a graph showing that, with learning over days, neural activity patterns are further from the speed limit and contribute to greater progress.

FIG. 8A-8C are graphs showing that the results produced using the method of the present invention do not depend on specific definitions of the speed limit.

FIG. 9 is a graphical depiction of the progress metric used to assess the behavioral impact of each neural activity pattern.

FIG. 10A is a graphic depiction showing that new neural activity patterns can be inside or outside of the intrinsic manifold. FIG. 10B is a graphic depiction showing that neural activity patterns can be decomposed into an inside-manifold component and an outside-manifold component.

FIG. 11A is a graph showing that subjects learn using both inside-manifold and outside-manifold strategies for a given OMP mapping. FIG. 11B is a graph showing that learning can arise from both outside-manifold and inside-manifold strategies for different targets.

FIG. 12 is a graph showing that subjects learn using both helpful and unhelpful inside-manifold strategies and helpful and un-helpful outside-manifold strategies.

### SUMMARY OF THE INVENTION

Existing neural interfaces rely on physiological control signals generated by the nervous system without any additional training. The incremental training procedure disclosed herein trains users to generate new patterns of physiological control signals. These new patterns can be leveraged for rehabilitation, to augment the pre-existing capabilities of the subject or to control physical objects.

The invention is described herein in terms of teaching a subject to produce new neural activity patterns. However, as may be realized, the methods of the invention may also be used to teach a subject to produce any new pattern of physiological control signals, for example, muscle activity, eye movements, sounds, etc.

The invention comprises a method of teaching a subject to produce new neural activity patterns that are outside of the intrinsic manifold. The specific neural activity pattern that the subject is trained to produce represents a desired OMP mapping of a neural activity pattern to a specific behavior. The subject is first taught the specific behavior using neural activity patterns representing an intuitive mapping to the behavior (i.e., a neural activity pattern within the intrinsic manifold). The subject is then provided with a series of mappings that incrementally diverge the neural activity pattern from the intuitive mapping and converge the neural activity pattern to the desired OMP mapping. Once the subject learns the behavior at each incremental mapping, the subject is provided with the next incrementally divergent mapping, until the subject is able to accomplish the desired behavior using the desired OMP mapping.

In one embodiment of the invention, a subject monkey moves a computer cursor from the center of a screen to one of eight peripheral targets (displayed one at a time) by volitionally modulating the activity of a population of ~90 neural units recorded with a multielectrode array implanted in the arm region of primary motor cortex of the monkey's brain (the BCI). The recorded neural units are a representative subset of all neurons involved in the learning. The method proceeds by presenting the monkey with an “intuitive mapping” that relates neural activity patterns to cursor velocities in a way that provides proficient control without requiring the subject to learn. The subject is then presented with a novel mapping from a neural activity pattern to cursor velocity.

To facilitate having the subject monkey learn the OMP mapping, the incremental paradigm explained above can be used. Multiday exposure to an OMP with no incremental training leads to inconsistent learning. Conversely, the incremental training approach is not effective within a single day. Learning proceeds in a manner that resembles skill learning: gradual improvement over the course of several days, with some dips and rebounds in performance. The dips and rebounds reflect some combination of the natural skill-learning process, motivation during a difficult task, and day-to-day recording instabilities of the subject.

Detecting the appearance of a new pattern of neural activity in the high-dimensional neural space is difficult because only a limited number of patterns are observed relative to the dimensionality of the space. Instead, the BCI framework is leveraged to look for the emergence of new neural activity patterns within the low-dimensional space specified by the BCI mapping. The patterns of neural activity observed before learning are defined as the “intuitive neural repertoire”. The ~90D neural activity patterns comprising the intuitive neural repertoire are then projected into the 2D velocity space defined by the OMP mapping. This defines the limits on cursor velocities the subject could produce through the OMP mapping if it only expressed neural activity patterns from within its intuitive neural repertoire. The limits on cursor velocities that the subject can produce is defined as the “neural speed limit.” Any cursor velocities observed after learning that exceed the neural speed limit must have arisen from neural activity patterns that are outside of the intuitive neural repertoire, and thus are new. Over the course of many days, subjects learned to move the cursor at velocities that exceeded the neural speed limit for many targets. The percentage of neural activity patterns that are new significantly increases over days, showing that the brain can generate new neural activity patterns when learning to perform a new skill.

It may be difficult to know if observed neural changes that accompany learning are directly responsible for the learned behavior. A BCI allows the assessment of the behavioral consequence of any given neural activity pattern. To assess the behavioral impact of each neural activity pattern, the component of cursor velocity in the direction of the target is measured and is referred to as “progress”. Higher progress indicates straighter and/or faster cursor movements. Over the course of a multiday experiment, progress improves. Increases in progress can be positively correlated with the emergence of more new neural activity patterns, which indicates that the subject learned to move the cursor faster and straighter to the target in part by producing new neural activity patterns.

The invention demonstrates that learning can proceed by the formation of new neural activity patterns that directly drive behavioral improvements. New neural activity patterns could arise from organized changes in neural firing rates that conform to the preexisting correlation structure, which are characterized as an inside-manifold change. Alternatively, new neural activity patterns could arise from changes in the correlations between the neurons, which are characterized as an outside-manifold change. To determine the extent to which the new patterns generated by the subjects resulted from firing rate changes or correlation changes, each neural activity pattern can be decomposed into an inside-manifold component and an outside-manifold component. This enables the determination of the inside-manifold and outside-manifold contributions to progress. The new neural activity patterns on the last day of a multiday learning experiment include patterns with both substantial inside- and outside-manifold contributions to progress. This means that the subjects learned to move the cursor to some targets by generating new neural activity patterns that were outside of the intuitive neural repertoire but within the manifold, whereas other targets were learned by generating new neural activity patterns that were both outside of the intuitive neural repertoire and outside of the intrinsic manifold. Thus, learning can occur by changing the correlation structure and also by changing firing rates in a manner that preserves the correlation structure. The brain can overcome the neural constraints imposed by the intrinsic manifold previously observed during single-day learning but doing so takes several days.

### Definitions

As used herein, the term “physiological control signals” is defined as physiological signals measured from the central or peripheral nervous systems, including the activities of neurons, muscles or other bodily features, that can be used as a control signals for actuation.

As used herein, the terms “pattern of neural activity” and “neural activity pattern” and are defined as the joint firing rate of a population of neural units measured during a brief time window.

As used herein, the term “neural unit” is a group of neurons providing various contributions to a reading at a specific electrode in a multielectrode array.

### DETAILED DESCRIPTION

To demonstrate the method of the present invention, a BCI paradigm was employed in which a subject demonstrates a desired behavior, namely, the movement of a computer cursor from the center of a screen to one of eight peripheral targets, by volitionally modulating the activity of a population of ~90 neural units recorded with a multielectrode array implanted in the arm region of primary motor cortex. The learning begins by presenting the subject with an “intuitive” mapping that relates neural activity to cursor velocities in a way that provides proficient control without requiring the subject to learn. A series of novel mappings from neural activity to cursor velocity are then presented to the subject. The novel mappings encouraged the formation of new neural activity patterns. The novel mappings are incrementally learned by the formation of new neural activity patterns, and those patterns directly drive the desired behavior.

Electrophysiology and Behavioral Monitoring—The neural activity is recorded from the arm region of the primary motor cortex (M1). In a proof of concept experiment, the neural activity patterns from two male rhesus monkey subjects (subject “L” and subject “N”) were recorded using 96-channel microelectrode arrays. The neural units were recorded as threshold crossings, where a threshold crossing was detected when the depolarizing phase of the voltage signal crossed a threshold of −3 times the root-mean-square voltage. The root-mean-square voltage of the signal was estimated on each electrode while the subjects sat calmly in a darkened room. For multi-day experiments, the threshold was set on day one and not adjusted again. Across multi-day experiments, 89.9±1.9 neural units were recorded for subject L, and 93.2±1.8 neural units were recorded for subject N. Within a given OMP experiment the number of recorded neural units was constant throughout. The number of threshold crossings were counted in non-overlapping 45-ms bins. The data were recorded between 18 and 41 months after array implantation for subject L and from 2.5 to 8 months after array implantation for subject N. Both arms of the subjects were loosely restrained throughout all experiments. While the subjects could move their forearms slightly and there was no restriction on wrist and hand movement, the arm movements during the experiments were minimal.

Behavioral Task—Throughout each experiment, the subjects performed an eight-target center-out task under brain-computer interface control. Each trial began with the cursor appearing at the center of the workspace. A peripheral target 125 mm away appeared simultaneously. Target locations were uniformly spaced 45 degrees apart around a circle. Targets were presented in a random (subject N) or a pseudo-random (subject L) order. Pseudo-random target presentation consisted of 8-trial blocks, in which the target location was randomly chosen without replacement, and the next block of trials began only after all eight targets were successfully acquired. The cursor was frozen at the center of the workspace for the first 300 ms of the trial. After this “freeze period” the cursor began moving under neural control. Target acquisition occurred when the cursor entered the target window (circle, radius=14 mm, subject L; 35 mm, subject N). If the target was successfully acquired within 7.5 s, the trial was deemed a success, the subject was given a reward, and the next trial began 200 ms later. If the target was not acquired within the 7.5 s allotted time, there was a 1.7 s timeout before the next trial.

Identifying the Intrinsic Manifold—The “intrinsic manifold” is defined as the low-dimensional space that describes the neural activity patterns generated by the subject prior to learning. To estimate it, data collected on day 1 was used, while the intuitive BCI mapping were calibrated. Factor analysis was used to describe each high-dimensional spike count vector in terms of a low-dimensional set of factors. Under factor analysis, factors are described by a standard normal distribution:

$${z}_{t} ∼ N ( 0 , I ) \qquad ( 1 )$$

where:
I is the identity matrix.

The neural activity is related to those factors by a normal distribution:

$${u}_{t} \vert {z}_{t} ∼ N ( Λ {z}_{t} , ψ ) \qquad ( 2 )$$

where:
uₜ∈ [symbol] ^{q×1} is the z-scored spike count vector across q simultaneously-recorded neural units at timestep t.

Each neural unit was z-scored separately. zₜ∈ [symbol] ^{p×1} is the set of p factors at timestep t. Λ∈ [symbol] ^{q×p} and

$$ψ ∈ {\sum }_{+}^{q}$$

(a diagonal q×q covariance matrix) were estimated using the expectation-maximization algorithm. The intrinsic manifold is defined as the column space of Λ, where each factor corresponds to a column of Λ. z-scoring ensures that the subjects are not required to produce firing rates that are outside of the observed physiological range of each neural unit to learn how to control the cursor while using the perturbed BCI mappings.

The dimensionality of the intrinsic manifold was set to be 10 for all experiments. The dimensionality was computed as the number of dimensions needed to explain 95% of the shared variance. This method returns a more reliable estimate of dimensionality, even though it tends to underestimate the “true” dimensionality. The underestimation occurs because it seeks only to explain 95% of the shared variance. Using this method, the multi-day experiments had a mean dimensionality of 8.4 (range: 5-11) across both subjects (computed using 3500-5500 time points for each session). (For each subject individually, dimensionalities were subject N: mean=9.6, range: 8-11; subject L: mean=7.4, range: 5-9). This distribution is consistent with the use of 10 dimensions.

Given the neural activity uₜ, the factors

$${\hat{z}}_{t} = E [ {Z}_{t} \vert {u}_{t} ] = {{Λ}^{T} ( Λ {Λ}^{T} + ψ )}^{- 1} {u}_{t} = β {u}_{t} \qquad ( 3 )$$

are extracted,
where:

$$β = {{Λ}^{T} ( Λ {Λ}^{T} + ψ )}^{- 1} .$$

A separate factor analysis model was fit for each experiment based on the neural activity recorded during the calibration trials.

Intuitive BCI Mappings—BCI mappings translated the neural activity into 2D cursor velocities vₜ using a Kalman filter. A Kalman filter consists of a state model, which describes how cursor velocity changes over time, and an observation model, which describes how observed neural activity relates to cursor velocity. The state model is:

$${v}_{t} \vert {v}_{t - 1} ∼ N ( A {v}_{t - 1} , Q ) \qquad ( 4 )$$

where:

$$A = I ; and \\ Q = 2 ( {m}^{2} / {s}^{2} ) \times I .$$

Q controls the smoothness of cursor velocities over time and was chosen based on the setting that yielded the best closed-loop performance. In a standard Kalman filter, the high-dimensional neural activity is directly translated into cursor velocities. The lower-dimensional factors were first estimated using Eq. (3) and then the factors were translated to cursor velocities. The observation model is:

$${\hat{z}}_{t} \vert {v}_{t} ∼ N ( C {v}_{t} + d , R ) \qquad ( 5 )$$

where:

$$C = {ℝ}^{10 \times 2} ; \\ d ∈ {ℝ}^{10} ; and \\ R ∈ {\sum }_{+}^{10} .$$

These parameters were fit using maximum likelihood to relate the factors and

intended cursor velocities (defined below) from the calibration trials at the start of each experiment. Taken together, the intuitive BCI mapping can be written as:

$${\hat{v}}_{t} = {M}_{1} {\hat{v}}_{t - 1} + {M}_{2} {u}_{t} + {m}_{0} {M}_{1} = A - KCA {M}_{2} = K β {m}_{0} = - Kd \qquad ( 6 )$$

where:
\hat{v}ₜ is the velocity used to move the cursor on the screen at time t; and
K is the converged Kalman gain.

M₁ temporally smooths the velocities, M₂ describes the high-dimensional neural activity in terms of the low-dimensional factors and relates the factors to the cursor velocity and m₀ is a constant offset.

Each experiment began with 80 trials to calibrate an intuitive BCI mapping. Calibration involved a mixture of passive observation of center-out cursor movements and closed-loop BCI cursor control, during which the subject's level of control over the cursor was gradually increased, until all assistance was removed, and the subject had full control. Calibration began with 16 trials (2 to each target) of the subject observing automatic center-out cursor movements straight to the target at a constant speed (0.15 m/s). Here, intended cursor velocity at each timestep was taken to be the observed cursor velocity (0.15 m/s) in the center-to-target direction. For the next 8 trials, the subject controlled the cursor using a mapping calibrated using the data from the 16 observation trials, but the cursor was restricted to move only along the center-to-target direction (velocity components perpendicular to the center-to-target direction were set to 0). The next 8 trials used a mapping calibrated from the previous 8 trials, and perpendicular velocity components were scaled by a factor of 0.2. The next 8 trials used a mapping calibrated from the previous 16 trials, and perpendicular velocity components were scaled by a factor of 0.4. This procedure was repeated for a total of 40 to 80 trials until the subject was given complete control of the cursor (perpendicular scale factor=1). All calibrations performed within this procedure defined intended cursor velocities to be straight to the target, with speeds taken from the cursor movements that were displayed to the subject. The subjects demonstrated proficient cursor control using the intuitive BCI mapping from the very first intuitive trial of each experiment, as evidenced by success rates (100%), fast acquisition times, and relatively straight cursor trajectories. The data from these 80 calibration trials were used to determine the intrinsic manifold.

Perturbed BCI Mappings—Perturbed BCI mappings alter the relationship between recorded neural activity and cursor velocities to induce learning. The OMP mappings are of the same general form as the intuitive mapping (Eq. (6)). OMP mappings modify an intuitive BCI mapping by permuting the elements of uₜ (that is, the ~90 neural units) before passing it into factor analysis. This changes the relationship between the neural activity and factors, but preserves the relationship between the factors and cursor velocity. The impact of this manipulation is that it encourages the subject to change the way in which neurons co-vary to restore proficient cursor control. In contrast, to generate a WMP mapping, the ten factors were permuted before passing them to the Kalman filter. This manipulation changes the relationship between the factors and cursor velocity, but preserves the relationship between the neural activity and the factors. An OMP mapping can be described in terms of the high-dimensional neural activity by:

$${\hat{v}}_{t} = {M}_{1} {\hat{v}}_{t - 1} + {M}_{2}^{OMP} {u}_{t} + {m}_{0} \qquad ( 7 )$$

where:

$${M}_{2}^{OMP} = {M}_{2 {η}_{OM}} ;$$

and
η_{OM} is a q×q permutation matrix defining the outside-manifold perturbation (where q indexes the neural channels).

The chosen OMP mappings were not orthogonal to the manifold, as this would result in essentially zero cursor movement, at least initially. Rather, each mapping was selected to be difficult enough to require substantial learning but not so difficult that the subject would give up.

Behaviorally, these perturbations had complex effects on cursor movements, which cannot be expressed as pure visuomotor rotations or gains. Before learning, the effects of a typical perturbation can be approximately summarized by a combination of per-target velocity rotations and speed scalings. Because the perturbations were implemented in high-dimensional space, these rotations and scalings need not be consistent across movement directions and speeds (as they would be in the case of a pure visuomotor rotation or gain). Perturbations often affected movement speeds more profoundly along one movement direction than along the perpendicular direction.

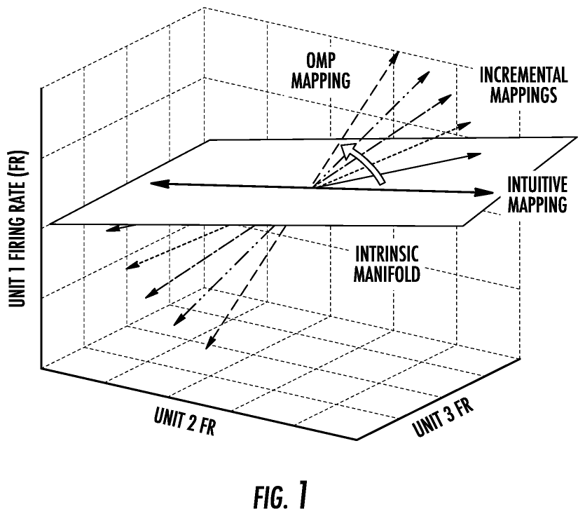

Incremental Training Paradigm—An incremental training paradigm was developed to facilitate learning. The incremental training paradigm consisted of giving the subject a series of incremental mappings which were successively further from the intrinsic manifold and successively closer to the full OMP mapping, to guide the subject through the neural space to the new patterns of neural activity requested by the OMP mapping. As shown in FIG. 1, the incremental mappings spanned the neural space between the intuitive mapping and the OMP mapping. Note that, for simplicity, FIG. 1 shows the incremental mappings for only 3D of the ~90D neural units. The incremental mappings are a weighted combination of the intuitive mapping and the OMP mapping, such that:

$${M}_{2}^{incremental} = ( 1 - \frac{a}{5} ) * {M}_{2} + ( \frac{a}{5} ) * {M}_{2}^{OMP} \qquad ( 8 )$$

where:
a=1, 2, 3, 4, 4.5.

Incremental step 1 is closer to the intrinsic manifold and should be easier to learn to control and incremental step 4 is closer to the OMP mapping and is expected to be more difficult to learn to control. The inclusion of the additional half step (a=4.5) was made to increase the subjects' willingness to continue to attempt the task. The criteria for incrementing the mapping are described below. Note that, while 5 steps are specified in the exemplary embodiment, any number of incremental steps could be used.

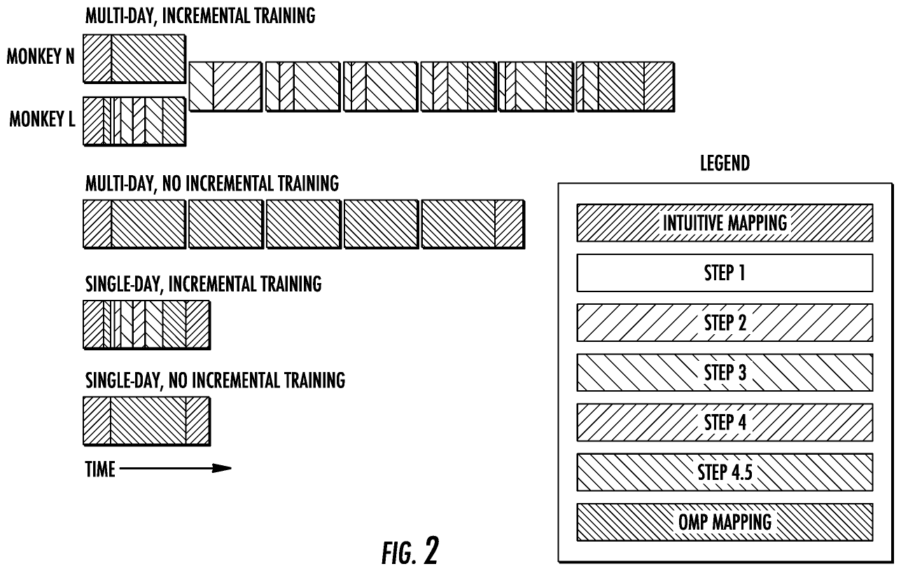

Task Flow—Each experiment began with a calibration block during which the intrinsic manifold was identified and the parameters of the intuitive mapping determined. FIG. 2 shows the training scheme used for single- and multi-day experiments with incremental training and no incremental training. Each block indicates a day. The subjects used the intuitive mapping for 206±56 trials (subject L) and 193±3.68 trials (subject N). One of four possible OMP experiment types were then run, as shown in FIG. 2: multi-day, incremental training (subject L: n=9; subject N: n=6); multi-day, no incremental training (subject L: n=6; subject N: n=5); single day, incremental training (subject L: n=12; subject N: n=15); single day, no incremental training (subject L: n=13; subject N: n=8). Single day WMP experiments are also included (subject L: n=11; subject N: n=11).

A total of 51 experiments were conducted with subject L and 45 experiments with subject N. The task for each experiment type is detailed below. The number of each type of experiment is summarized in Table 1:

TABLE 1

| | Multi-day | Multi-day | Single-day | Single-day | |
|---|---|---|---|---|---|
| | OMP, | OMP, no | OMP, | OMP, no | Single- |
| | incremental | incremental | incremental | incremental | day |
| Monkey | training | training | training | training | WMP |
| L | 9 (7 to 16 | 6 (3 to 7 | 12 | 13 | 11 |
| | days) | days) | | | |
| N | 6 (6 to 11 | 5 (5 days) | 15 | 3 | 11 |
| | days) | | | | |

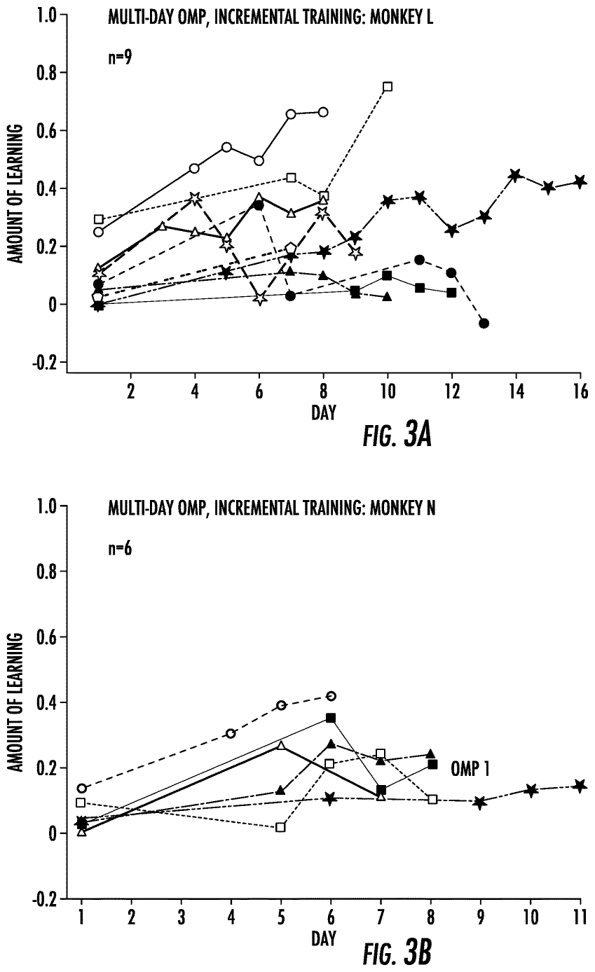

Multi-day OMP (With Incremental Training)—FIG. 3A and FIG. 3B show Learning curves for all multi-day OMP (with incremental training) experiments for subject L and subject N, respectively. The amount of learning (circles) is shown only for days on which the full OMP was presented to the subject. Each line indicates a unique OMP mapping. The procedures for multi-day (with incremental training) experiments differed somewhat for the two subjects.

For subject N, on day 1 the intuitive mapping block was followed by that experiment's full OMP mapping (434±81 trials). Day 2 began with 40 trials of the full OMP mapping before beginning the incremental training with the incremental step 1 mapping. The mapping was incremented when the subject reached a success rate of 80% or greater over the prior 40 trials and target acquisition times appeared to asymptote according to visual inspection by the experimenter. On subsequent days, subject N began with an incremental mapping (usually step 3) and incremental training continued in the same way. After arriving at the full OMP mapping, the experiment continued for another 2 to 3 days (2.8±1.2 days). At the end of the final day, the intuitive mapping from day 1 was re-introduced for a washout block (356±130 trials).

For subject L, day 1 of a multi-day (with incremental training) experiment proceeded in the same manner as a single day (with incremental training) experiment (as described below) without a washout block. On subsequent days, the subject began with an incremental mapping (usually step 3) and incremental training continued. That is, the full OMP mapping was not re-presented at the start of day 2, as it was for subject N. There were no objective criteria for incrementing to the next mapping for subject L. Instead, the mapping was incremented when subject L showed good performance as judged by the experimenter. This procedure was adopted in part because subject L was more likely to give up when the task was difficult, and it was felt that a fixed rule might actually disrupt learning. After reaching the full OMP mapping, the experiment continued for another 4.5±3.1 days. At the end of the final day, the intuitive mapping from day 1 was re-introduced for a washout block (425±283 trials).

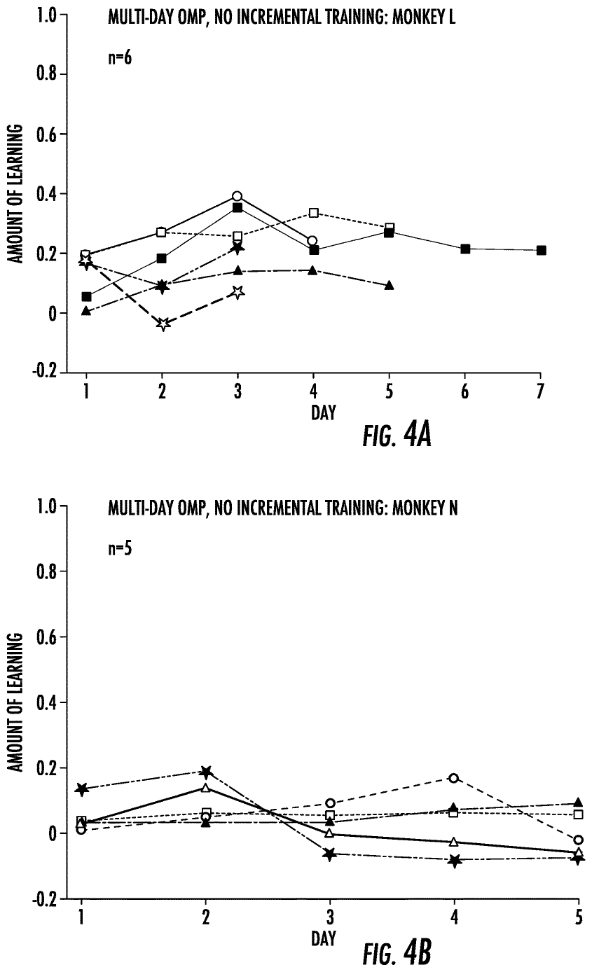

Multi-day OMP (Without Incremental Training)—FIG. 4A and FIG. 4B show the results of all multi-day OMP (without incremental training) learning curves for subject L and subject N respectively. In this type of experiment, on day 1 the intuitive mapping block was followed by a full OMP mapping block for 250 to 815 trials (L: 563±226 trials; N: 440±90 trials). The first day ended with no washout block. The same OMP mapping was presented for 3 to 7 days (subject L: 4.5±1.5 days; subject N: 5±0 days). On subsequent days the subject saw only that experiment's full OMP mapping, and the subject had at least 400 trials (subject L: 738±257 trials; subject N: 443±139 trials) of exposure to it each day. At the end of the final day of each multi-day OMP (without incremental training) experiment the intuitive mapping from day 1 was re-introduced for a washout block (699±205 trials).

Single-day OMP (With Incremental Training)—In this type of experiment, the intuitive mapping block was followed by a 40 to 160 trial block with that day's full OMP mapping. Then incremental training began. The subject saw each incremental mapping for the same number of trials regardless of performance: Incremental step 1 for 40 trials, incremental step 2 for 40 trials, incremental step 3 for 80 trials, incremental step 4 for 80 trials, incremental step 4.5 for 120 or 160 trials, and then the full OMP mapping for at least 160 trials. These block sizes were chosen to balance the number of trials it took for the subject to improve performance and to minimize the number of rewards the monkey received when the task required less learning (i.e., when the 8 incremental mapping was closer to the intuitive mapping). Following the full OMP mapping block, the intuitive mapping was re-introduced for a washout block (342±99 trials).

Single-day OMP (Without Incremental Training)—In this type of experiment, the intuitive mapping block was followed by a block with that day's full OMP mapping for 116 to 600 trials (subject L: 365±85 trials; subject N: 575±70 trials). Following the OMP mapping block, the intuitive mapping was re-introduced for a washout block (206±83 trials).

Single-day WMP—In this type of experiment, the intuitive mapping block was followed by a WMP mapping block for 189 to 800 trials (subject L: 331±98 trials; subject N: 616±58 trials). Following the WMP mapping block, the intuitive mapping was re-introduced for a washout block (subject L: 167±60 trials; subject N: 334±108 trials).

Amount of Learning—For each experiment, the amount of learning (AoL) during OMP control was computed, which provides a measure of the extent to which learning recovered intuitive levels of performance subsequent to the introduction of the BCI perturbation. The metric is based on reward rate (i.e. the number of successful trials per unit time when the cursor is under the subject's control) and accounts for the reward rate with the intuitive mapping, as well as the potential learning possible given the difficulty of the perturbed mapping. Reward rate (RR) was computed for a sliding window of 40 consecutive trials. Then, we quantified the amount of learning as:

$${AoL}_{T} = \frac{{RR}_{T}^{OMP} - {RR}_{1}^{OMP}}{{RR}^{intutive} - {RR}_{1}^{O M P}} \qquad ( 9 )$$

where:
RR^{intuitive} is the reward rate calculated for the 40 trials with the intuitive mapping just before the perturbation is introduced; and

$${RR}_{T}^{OMP}$$

is the reward rate with the OMP mapping calculated for a sliding window of 40 trials beginning at trial T after the OMP is introduced.

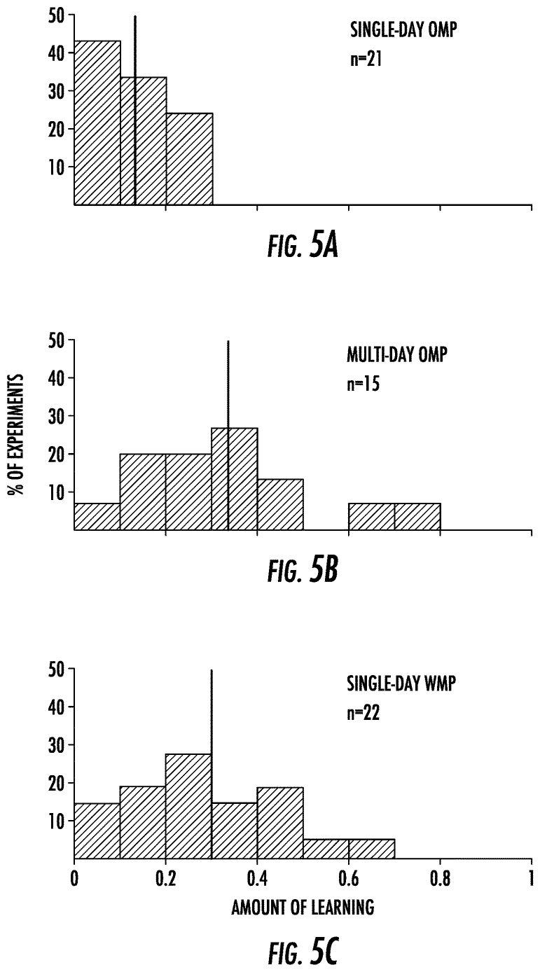

A value of 1 reflects performance that was fully restored to intuitive levels of control, and a value of 0 reflects no performance improvement. This was chosen because it better captures the learning across all targets when the subjects had a particularly low success rate to a subset of the targets, which was common for the OMP mappings studied here. FIG. 5A-C shows histograms quantifying the amount of learning for single-day OMP (FIG. 5A), multiday OMP (with incremental training) (FIG. 5B) and single-day WMP (FIG. 5C) experiments. An amount of learning of 1 indicates complete learning, and a value of 0 indicates no learning. Vertical lines indicate the mean of each distribution.

For single day experiments, AoL=max_{T}(AoL_{T}). For multi-day experiments, the AoL for each T on each day was found. The AoL values reported in the histograms in FIG. 5 are the maximum AoL across all days. Thus, it reflects the best 40 consecutive trials on the best day, to showcase the learning. The results were qualitatively similar when the best 40 trials were considered from the first day the subject saw the full OMP mapping after incremental training or when the best 40 trials were considered from the last day the subject saw that OMP mapping. Because the effects were robust to these choices, this shows that the learning is not trivially dependent on the particular trials chosen.

Neural Analyses—Because the goal was to characterize changes in neural activity that facilitate learning, the trials that showed the greatest improvement in behavioral performance were focused. For the neural analyses, the successful trials from the 40 consecutive perturbation trials that showed the best AoL on each day were analyzed. Failed trials interspersed within that block of 40 trials were not analyzed because it is difficult to determine whether the animal was actively engaged in the task during failed trials.

Defining New Neural Activity Patterns With a Speed Limit—To look for the emergence of new neural activity patterns, a “speed limit” is defined. To define the speed limit each intuitive neural activity pattern was projected through the OMP mapping:

$${\hat{v}}_{t} = {M}_{2}^{OMP} {u}_{t} + {m}_{0} \qquad ( 10 )$$

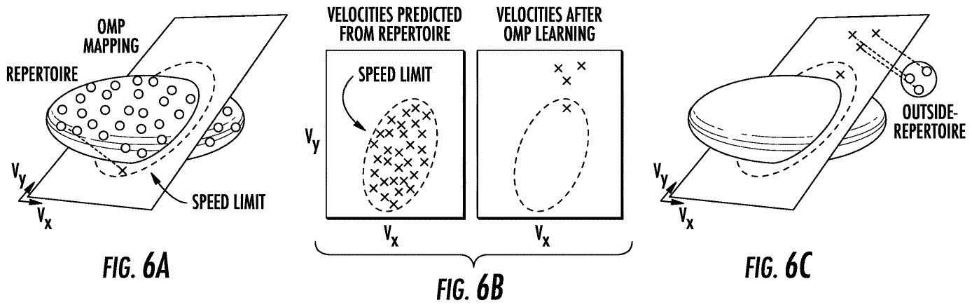

FIGS. 6A-6C schematically show the technique used to identify outside-repertoire activity patterns and show that new neural activity patterns emerge with long-term BCI learning. The OMP maps ~90D neural population activity patterns to 2D cursor velocities. Here 30 neural activity patterns are illustrated and a 2D OMP mapping are illustrated. FIG. 6A shows the neural activity patterns (dots) generated by the subject while using the intuitive mapping define an intuitive neural repertoire (ellipsoid). Each neural activity pattern maps to a cursor velocity (X, as one example) through the OMP mapping plane. FIG. 6B (left) shows the velocities predicted from the intuitive neural repertoire (Xs) through the OMP mapping define a speed limit (dashed ellipse). FIG. 6B (right) shows that, after learning, cursor velocities are observed that exceed the speed limit (three Xs outside of ellipse). FIG. 6C shows that, if the subject produces cursor velocities that exceed the speed limit, those velocities were generated by neural activity patterns that lie outside of the intuitive neural repertoire and thus are new.

The impact of the neural activity patterns on the cursor kinematics are of particular interest and so do not include the previous timestep's velocity (i.e., M₁\hat{v}ₜ₋₁ in Eq. (6)). The 95% convex hull of the resulting velocities was computed. This convex hull is a 2D representation of the intuitive neural repertoire in the behaviorally-relevant space. The convex hull is the smallest polygon enclosing all the 2D points that also encloses all possible line segments between any two points within the polygon. To be robust to outliers, the outermost points were successively dropped until 5% of all the points had been excluded. Points nearest to the boundary of the hull were dropped in order from largest to smallest Mahalanobis distance from the centroid of all points in the 2D projection. Mahalanobis distances were computed relative to the covariance across all points in 2D.

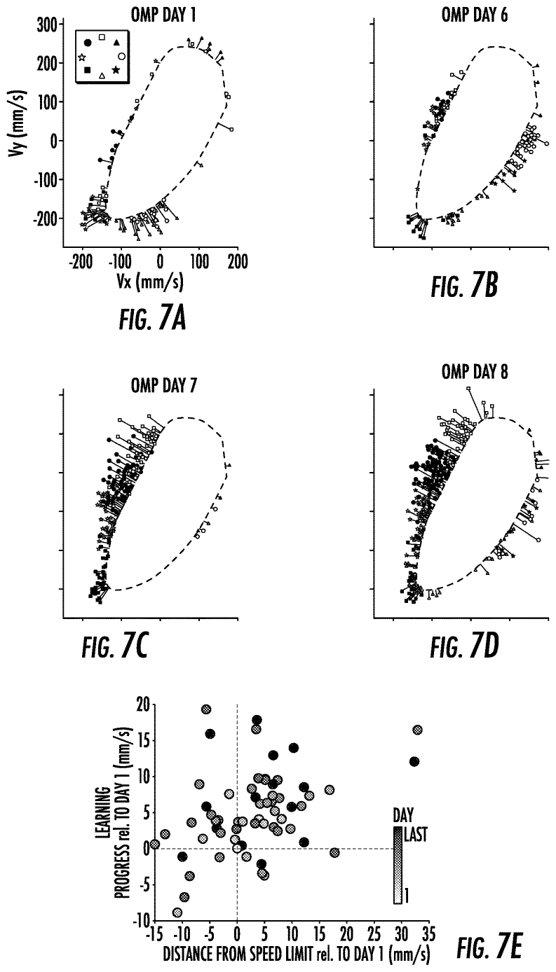

The convex hull is referred to as a “speed limit” because it represents the maximum speed in each direction that neural activity patterns within the intuitive neural repertoire would have produced under the OMP mapping. By definition, cursor speeds exceeding the speed limit could not have been achieved by the previously-observed neural activity patterns. Therefore, any neural activity patterns leading to cursor speeds exceeding the speed limit were classified as new. This is a conservative definition of new patterns in the sense that it does not capture patterns of neural activity outside the ~90D neural repertoire that map to speeds within the speed limit. The percentage of new patterns (e.g., FIG. 7D) is the ratio of new neural activity patterns to total neural activity patterns generated by the subject during the 40-trial block (successful trials only).

FIGS. 7A-7D show the use of the speed limit to detect new neural activity patterns. The speed limit is defined as the 95% convex hull of the velocities generated when the intuitive neural repertoire is mapped through the OMP. New neural activity patterns are defined as patterns that generate velocities which exceed the speed limit. With learning, more new neural activity patterns are observed. One possible interpretation of this result is that the velocities that exceed the speed limit arise from visiting those 5% of points outside the speed limit progressively more often. If this were case, the percentage of “new” neural activity patterns identified by the speed limit metric would be expected go up, but the distance of those points from the speed limit would be expected to remain unchanged. However, this possibility is not consistent with the data. Instead, FIGS. 7A-7D show the movement of the points progressively farther from the speed limit on days 1, 6, 7 and 8 respectively, showing that the points move progressively farther from the speed limit (gray dashed) during learning (i.e., the cursor velocities continue to get faster and more target-directed during learning). FIG. 7E is a graph showing that, with learning over several days, neural activity patterns are further from the speed limit, and they contribute to greater progress. For the multi-day OMP experiments, there is a significant correlation between the extent of the points beyond the speed limit and increases in progress (Pearson correlation coefficient r=0.40, p=0.0002). Each symbol is the average over all 8 targets from one day of one OMP experiment relative to day 1. Symbols are shaded to indicate the day within a given multi-day experiment.

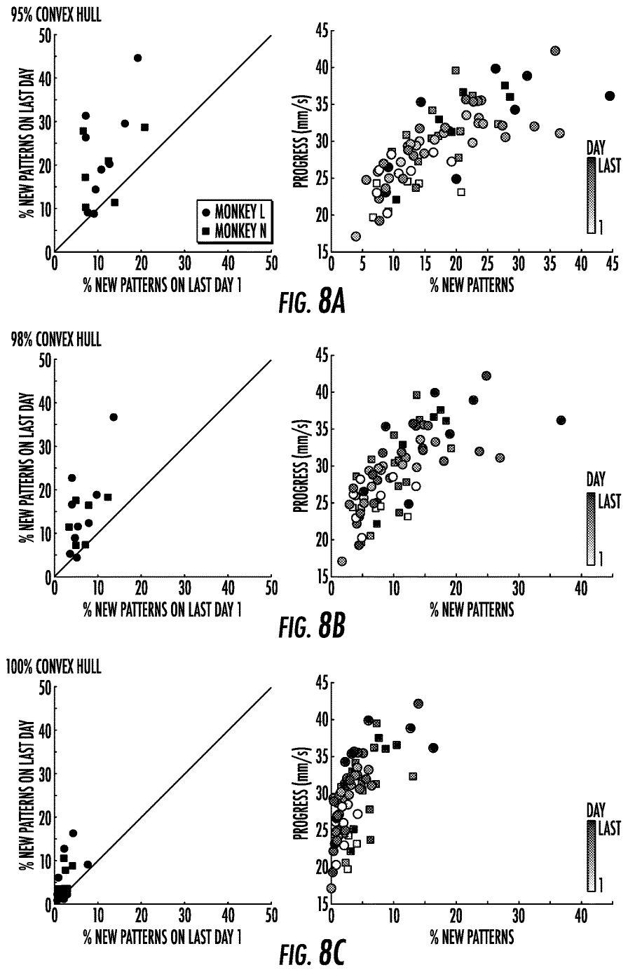

The 95% convex hull has been used to define the speed limit and to identify the emergence of new neural activity patterns. To ensure that the results did not depend on the percentage of points contained within the convex hull, the analyses for 98% and 100% convex hulls was repeated, with the results shown in FIGS. 8A-8C. The results were consistent across these definitions of the speed limit. (i.e., the results do not depend on the specific definition of the speed limit). The speed limits were defined as the 95% convex hull, shown in FIG. 8A, the 98% convex hull, shown in FIG. 8B and the 100% convex hull, shown in FIG. 8C.

Cursor Progress—To quantify the behavioral consequence of each neural activity pattern, the component of cursor velocity in the direction of the target at each timestep was measured. This metric is termed “progress”:

$${P}_{t} = [ \begin{matrix}cos {θ}_{t} \\ sin {θ}_{t}\end{matrix} ] * ( {M}_{2}^{OMP} {u}_{t} + {m}_{0} ) \qquad ( 11 )$$

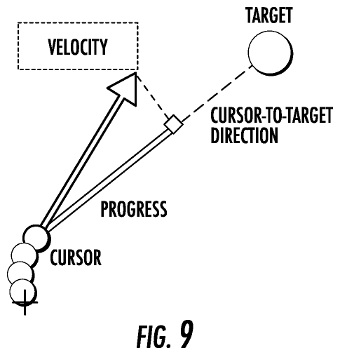

which is the projection of the single-timestep cursor velocity onto a unit vector in the cursor-target direction θₜ. This metric is depicted graphically in FIG. 9. While the AoL metric (i.e., reward rate) provides an overall metric of learning, the change in the progress metric provides a measure of the impact of each neural activity pattern on performance. Changes in progress provide a metric of per-target learning.

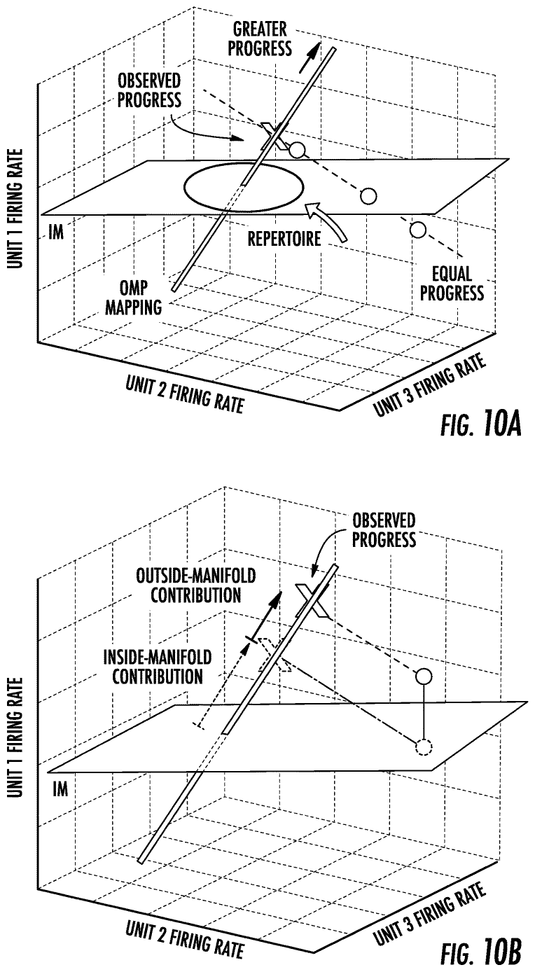

Outside-Manifold Contribution to Progress—New neural activity patterns can be inside or outside of the intrinsic manifold. FIG. 10A shows that a particular value of cursor progress can be produced by many different neural activity patterns (dashed line), including both patterns inside and outside of the manifold. In FIG. 10B, each neural activity pattern can be decomposed into an inside-manifold component and outside-manifold component. Correspondingly, observed progress (upper “X”) is the sum of inside-manifold contribution to progress and outside-manifold contribution to progress.

Correspondingly, the observed progress at each timestep is the sum of the inside-manifold contribution to progress and the outside-manifold contribution to progress, as depicted FIG. 10B. The progress that can be attributed to getting outside of the intrinsic manifold is computed according to:

$${P}_{t}^{outsideManifold} = {P}_{t}^{total} - {P}_{t}^{insideManifold} \qquad ( 12 )$$

where:

$${P}_{t}^{total}$$

is calculated from Eq. (11).

To calculate

$${P}_{t}^{insideManifold} ,$$

first, the orthogonal projection of each neural population activity pattern into the manifold was found:

$$orthogonal projection into manifold = {U}^{T} {u}_{t} \qquad ( 13 )$$

where:
uₜ is the z-scored spike count vector.

Because the spike counts have been z-scored, the mean of uₜ is 0. U contains the orthonormalized columns of Λ and is found using the singular value decomposition, such that Λ=USV^{T}. The column space of U defines the intrinsic manifold. Then, the representation of the orthogonal projection in the high-dimensional space was found:

$${\tilde{u}}_{t} = {UU}^{T} {u}_{t} \qquad ( 14 )$$

where:
ũₜ is the inside-manifold component of the population activity pattern.

Finally, the progress from this inside-manifold component of the spike count vector was computed:

$${P}_{t}^{insideManifold} = [ \begin{matrix}cos {θ}_{t} \\ sin {θ}_{t}\end{matrix} ] * ( {M}_{2}^{OMP} {\tilde{u}}_{t} + {m}_{0} ) \qquad ( 15 )$$

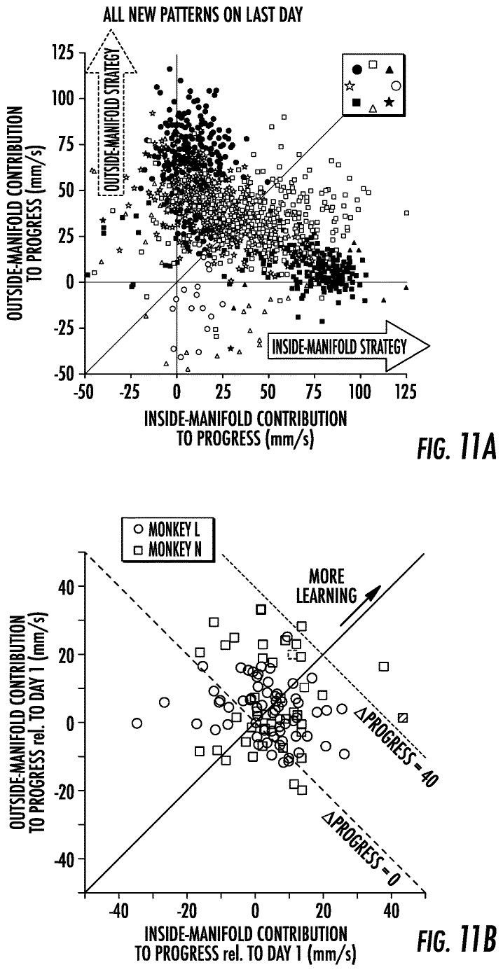

FIG. 11A shows the

$${P}_{t}^{insideManifold} and {P}_{t}^{outsideManifold}$$

for each of the new neural activity patterns expressed on the last day of OMP1. Some neural activity patterns produce large inside-manifold contributions to progress and small outside-manifold contributions to progress. Other neural activity patterns produce small inside-manifold contributions to progress and large outside-manifold contributions to progress. To further show that learning is not constrained by the intrinsic manifold, in FIG. 11B, the change in the mean

$${P}_{t}^{insideManifold}$$

and mean

$${P}_{t}^{outsideManifold}$$

across all neural activity patterns produced for each target relative to day 1 are shown. For this calculation all of the neural activity patterns expressed for each target were included, not only the new neural activity patterns. For many targets learning occurs by increasing the

$${P}_{t}^{outsideManifold} ,$$

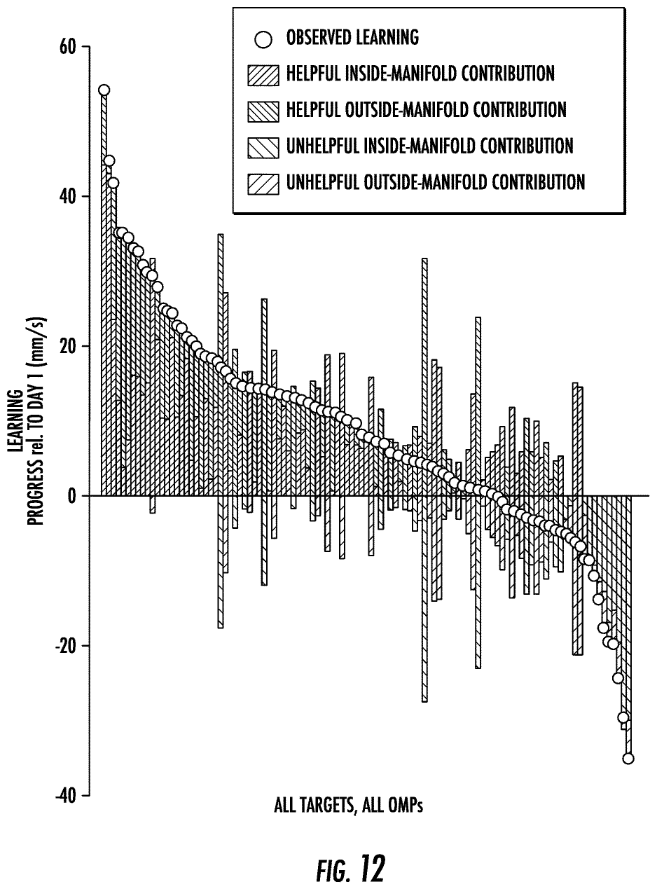

thus demonstrating that learning can be driven by producing new neural activity patterns outside the intrinsic manifold. Note that even for some targets that show good learning, the inside-manifold or outside-manifold component can be unhelpful, but then overcome by helpful progress in the other component, as shown in FIG. 12.

Embodiments—Various embodiments of the invention may include a computer comprising a processor and memory, the memory containing software for execution by the processor, the software implementing the described process of incremental training. The computer may further comprise a display for displaying, in one embodiment, the targets and the cursor. In other embodiments, the display may show other information regarding the desired behavior. The computer may be coupled to a sensor for receiving the physiological control signals. In one embodiment, the sensor may be a BCI and the software may receive and analyze signals from the BCI representing the neural activity patterns. The neural activity patterns may be translated into cursor movements by the software and displayed on the display.

As previously indicated, the invention is described herein in terms of teaching a subject to produce new neural activity patterns, but is equally applicable to teaching the subject to produce any new pattern of physiological control signals, for example, muscle movements, eye movements, sound patterns, etc. to control a desired behavior. Herein, the desired behavior is simplified in the described experiments because of the limited intelligence of the subject monkeys, who need to first be taught the desired behavior before new patterns of physiological control signals can be taught to produce the desired behavior. If implemented in human subjects, the desired behavior could simply be verbally (or otherwise) communicated to the subjects, thereby simplifying the process. The new patterns of physiological control signals taught in accordance with the methods disclosed herein could be used to as a signal to actuate or control physical objects in human subjects.

1. (canceled)

2. A system for training a subject to create new patterns of physiological control signals in the brain of the subject, comprising:
a brain-computer interface comprising a multielectrode array coupled to the subject for receiving from the subject signals representing a neural activity pattern of a population of neural units;
a processor, coupled to the brain-computer interface;
software that, when executed on the processor, provides the functions of:
recording a neural activity pattern received from the brain-computer interface, the neural activity pattern representing a mapping to a desired behavior inside an intrinsic manifold of the subject;
determining a sequence of mappings of neural activity patterns that incrementally diverge from the neural activity pattern inside the intrinsic manifold to a neural activity pattern outside the intrinsic manifold that produces the desired behavior;
iteratively performing the steps of:
exposing the subject to a current mapping in the sequence of mappings;
collecting, from the subject, via the brain computer interface, a neural activity pattern as the subject attempts the desired behavior;
translating the collected neural activity pattern to a resulting behavior; and
showing the resulting behavior using the current mapping;
wherein the subject moves from the current mapping in the sequence to a next mapping in the sequence when the subject produces the desired behavior using the neural activity pattern of the current mapping.

3. The system of claim 2 wherein the neural units are recorded when a depolarizing phase of a voltage signal at electrode of the multielectrode array crossed a threshold with respect to a reference voltage.

4. The system of claim 2 wherein the software performs the further function of:
deriving a low-dimensional representation of the neural activity pattern from the high-dimensional neural activity pattern.

5. The system of claim 2 wherein the population of neural units in the recorded neural activity pattern is a representative subset of all neural units involved in producing the desired behavior.

6. The system of claim 2 further comprising:
a physical object;
wherein the desired behavior is manipulating the physical object in a specific manner.

7. The system of claim 5 wherein the neural activity patterns represent muscle movements.

8. The system of claim 2 wherein the neural activity patterns represent the activity of a plurality of neural units.

9. The system of claim 8 wherein each incremental neural activity pattern is produced when the subject alters the co-variance of the neural units.

10. The system of claim 2 wherein each mapping in the sequence of mappings perturbs the neural activity pattern in a linear manner from mapping to mapping.

11. The system of claim 2 wherein each mapping in the series of mappings is a weighted combination of the neural activity pattern inside the intrinsic manifold and the neural activity pattern outside the intrinsic manifold.

12. The system of claim 2 wherein the intrinsic manifold is a low-dimensional space derived from the high-dimensional space of the of the neural activity pattern prior to the training.

13. The system of claim 2 wherein the desired behavior is the movement of a 2D cursor toward a target displayed on a screen.

14. The system of claim 13 wherein the neural activity patterns are translated to a cursor velocity in the direction of the target.

15. The system of claim 13 wherein new neural activity patterns are identified by cursor velocities that exceed a speed limit, wherein the speed limit is the maximum cursor velocity in each direction that neural activity patterns in the intrinsic manifold produce under the desired mapping.

16. The system of claim 13 wherein a behavioral consequence of each neural activity pattern in the sequence of mappings is indicated by a progress metric, the progress metric measuring a component of cursor velocity in a direction of the target at each timestep.

17. The system of claim 16 wherein the progress at each timestep comprises a contribution of an inside-manifold component and an outside-manifold component.

18. The system of claim 2 wherein the subject is exposed to the next mapping in the sequence of mappings when the subject produces the desired behavior a predetermined percent of the time within a predetermined number of trials.

19. The system of claim 2 wherein progress of the training is indicated by a metric representing a number of successful trials per unit time.
